(19) United States
(12) Patent Application Publication
(10) Pub. No.: US 2026/0224480 A1
(43) Pub. Date: Aug. 6, 2026

(54) SYSTEM FOR ORAL DELIVERY OF AN ACTIVE INGREDIENT

(71) Applicant: Philip Morris Products S.A., Neuchatel (CH)

(72) Inventors: Frédéric LAVANCHY, Chavornay (CH); Fabiana SPADARO, Lausanne (CH)

(73) Assignee: Philip Morris Products S.A., Neuchatel (CH)

(21) Appl. No.: 19/150,091

(22) PCT Filed: Jan. 26, 2024
(86) PCT No.: PCT/EP2024/051991

§ 371 Date: Jul. 22, 2025

(30) Foreign Application Priority Data
Jan. 27, 2023 (EP) 23153776.2

(51) Int. Cl. A61K 9/00; A61J 1/03; A61K 9/20; A61K 31/00; A61K 31/444; A61K 31/465; A61K 31/522

(52) U.S. Cl. CPC A61K 9/006; A61J 1/035; A61K 9/2018; A61K 9/2054; A61K 9/2086; A61K 31/444; A61K 31/465; A61K 31/522; A61K 31/658

(57) ABSTRACT

A system for oral delivery of an active ingredient is provided, the system including: a mucoadhesive layer including a mucoadhesive and an active ingredient; and a backing layer extending over the mucoadhesive layer, the backing layer including a bulk agent, the active ingredient being selected from the group consisting of nicotine, caffeine, anatabine, at least one cannabinoid such as tetrahydrocannabinol, and a combination thereof, the system being orally dissolvable, and the bulk agent being selected from the group consisting of at least one mono-, di-, and/or polysaccharide, at least one sugar alcohol, and a combination thereof.

### FIELD OF THE INVENTION

The present invention relates to a system for oral delivery of an active ingredient such as nicotine, caffeine or cannabinoids. The system is waste-free and achieves efficient transfer of the active ingredient through the oral mucosa of a user.

### BACKGROUND OF THE INVENTION

Various products for oral delivery of active ingredients are known. Examples include, lozenges, fast dissolving films, chewing gums and pouches. A disadvantage of such products is that the active ingredient contained therein is mainly delivered to the blood by absorption through the gastro-intestinal tract after swallowing saliva, while only a small portion is transferred through the oral mucosal membranes. Since many active ingredients are destroyed by the conditions in the gastro-intestinal tract of the user, the known products do not achieve efficient systemic delivery of the active ingredient.

WO 2011/137491 A1 describes a transmucosal delivery device comprising a solid core comprising an active ingredient, a bioadhesive layer, and a backing layer. A surface of the core is exposed and the device can be placed on the oral mucosal surface with the exposed surface and the bioadhesive layer in contact with the oral mucosal surface. The bioadhesive layer does not contain an active ingredient. A disadvantage of the above device is that it must have a large area to allow for both sufficient transfer of the active agent through the exposed area of the core and sufficient contact of the bioadhesive layer with the mucosa so that the device can stay in place. This makes the device inconvenient to use.

WO 00/62764 relates to a gum pad for delivery of medication to mucosal tissues. The gum pad is a laminate composed of a backing layer which is configured to be held in place on the gums in the mouth, an intermediate layer containing medication therein, and a semi-permeable outer layer. During use, saliva enters the semi-permeable outer layer and dissolves the medication in the intermediate layer which then diffuses outwardly to the mucosal tissue in the mouth. A disadvantage of the above gum pad is that absorption of the medication through the gastro-intestinal tract after swallowing saliva is not effectively prevented. Furthermore, the backing layer of the gum pad is made of a material which does not dissolve in the oral cavity. Thus, the gum pad must be removed from the mouth and discarded after use and is therefore not waste-free.

US 2021/0000161 A1 describes an orally dissolvable film containing a nicotine activator, a binder and a plasticizer. The film quickly disintegrates in the oral cavity of the user and thus a significant proportion of available nicotine is swallowed.

There is thus a need for systems which do not generate waste and efficiently deliver the active ingredient through the oral mucosal membranes of a user.

### OBJECT OF THE INVENTION

A object of the present invention is to provide a system for delivery of an active ingredient which achieves efficient delivery of the active ingredient. Another object of the invention is to provide a system which is completely dissolved after the active ingredient has been absorbed and thus does not generate any waste.

The system for oral delivery described below satisfies the above aims.

### SUMMARY OF THE INVENTION

In one aspect, the invention relates to a system for oral delivery of an active ingredient, the system comprising: a) a mucoadhesive layer comprising a mucoadhesive and an active ingredient and b) a backing layer extending over the mucoadhesive layer, said backing layer comprising a bulk agent and optionally a flavoring agent, wherein the active ingredient is selected from the group consisting of nicotine, caffeine, anatabine, at least one cannabinoid such as tetrahydrocannabinol, and a combination thereof, wherein the delivery system is orally dissolvable, and wherein the bulk agent is selected from the group consisting of at least one mono-, di-, and/or polysaccharide, at least one sugar alcohol and a combination thereof.

In a preferred embodiment, the backing layer further comprises a binder. In a further preferred embodiment, the backing layer further comprises a dissolution enhancer. In a further preferred embodiment, the mucoadhesive layer further comprises a penetration enhancer. In a further preferred embodiment, the backing layer is free of active ingredient. In a further preferred embodiment, the weight ratio of active ingredient to mucoadhesive in the mucoadhesive layer is about 1:1 to about 1:1000. In a further preferred embodiment, the weight ratio of active ingredient in the mucoadhesive layer to the weight ratio of active ingredient in the backing layer is about 1:1 to about 1000:1, preferably about 10:1 to about 500:1, more preferably about 50:1 to about 300:1, and most preferably about 100:1 to about 250:1. In a further preferred embodiment, the mucoadhesive is selected from the group consisting of gelatin, chitosan, (sodium) carboxymethyl cellulose, hydroxypropylmethyl cellulose, polyvinyl alcohol, gellan gum, agar, agarose, and a combination thereof. In a preferred embodiment, the active ingredient is nicotine.

In a further aspect, the invention relates to a dosage form comprising a system according to the invention. In a preferred embodiment, the dosage form is a tablet. In another aspect, the invention relates to a blister pack comprising the dosage form (e.g. tablet) according to the invention.

### DESCRIPTION OF THE FIGURES

FIG. 1: A system for oral delivery according to one embodiment of the present invention.

FIG. 2: A system for oral delivery according to another embodiment of the present invention.

### DETAILED DESCRIPTION OF THE INVENTION

The present invention is to provide a system for delivery of an active ingredient which achieves efficient delivery of the active ingredient and is waste-free.

The system for oral delivery of an active ingredient comprises: a) a mucoadhesive layer comprising a mucoadhesive and an active ingredient and b) a backing layer extending over the mucoadhesive layer, said backing layer comprising a bulk agent and optionally a flavoring agent, wherein the active ingredient is selected from the group consisting of nicotine, caffeine, anatabine, at least one cannabinoid such as tetrahydrocannabinol, and a combination thereof, wherein the delivery system is orally dissolvable, and wherein the bulk agent is selected from the group consisting of at least one mono-, di-, and/or polysaccharide, at least one sugar alcohol and a combination thereof.

During use, the system for oral delivery according to the invention is placed in the user's mouth such that the mucoadhesive layer is in contact with the oral mucosa. The mucoadhesive layer can securely adhere to the oral mucosa, thereby avoiding swallowing of the active ingredient. The backing layer covers the surface of the mucoadhesive layer opposite the surface which is in contact with the oral mucosa. The backing layer thus prevents contact of the active ingredient in the mucoadhesive layer with saliva and ensures that the active ingredient is not swallowed. Rather, the active ingredient is advantageously absorbed directly into the bloodstream directly through the oral mucosa.

Furthermore, the system for oral delivery according to the invention is orally dissolvable and thus does not generate waste.

### Definitions and Terminology

All technical and scientific terms used hereafter carry the commonly understood meaning of the word unless defined otherwise.

Unless defined otherwise, any feature(s) within any aspect or embodiment of the invention may be combined with any feature(s) within any other aspect or embodiment of the invention, and the skilled person understands such combination as being encompassed in the original disclosure of the present application. This also applies to the possibility of combination of elements from lists of species within definitions of different broader terms with one another, said possibilities of individualized combination being expressly intended and within the direct and unambiguous disclosure of the present application. This also applies in particular to all embodiments described within the section relating to system and their components, in respect of other aspects, e.g. dosage forms of those systems. This also applies in particular, but not exclusively, to endpoints of ranges disclosed herein. For instance, if a given substance is disclosed as existing in a composition in a concentration range of X-Y % or A-B %, the present application is to be understood as explicitly disclosing not only the ranges X-Y % and A-B %, but also the ranges X-B %, A-Y % and, in as far as numerically possible, Y-A % and B-X %. Each of these ranges, and range combinations, are contemplated, and are to be understood as being directly and unambiguously disclosed in the present application.

Similarly, it is also to be understood that section headings as used throughout the present application are merely for organizational purposes, and do not confer exclusive character to the disclosure pertaining to aspects or embodiments of the invention mentioned in a particular section heading. Section headings thus do not exclude the combination of the disclosure within any one section heading to a corresponding or analogous context provided under another section heading. Indeed, such intra-section disclosures are explicitly within the disclosure of the present application. As used herein, each of the terms “comprising', “having” and “containing”, including grammatical variants thereof, are meant in a non-exhaustive sense to mean “including”, but not necessarily “composed of”, and does not exclude elements in addition to those explicitly recited as being present. As used herein, then, the terms “comprising”, “having” and “containing”, and grammatical variants thereof, indicate that components other than those explicitly recited may, but need not, be present. As such these terms include as a limiting case embodiments in which no other elements than those recited are present, e.g. in the commonly accepted sense of “consisting of”.

As used herein, the phrase “consisting of”, and grammatically related variants thereof, means that no other elements are present than those recited. In standing with the above definition of “comprising” (and grammatically and semantically related terms), the term “consisting of” therefore denotes a limiting scenario within the meaning of “comprising”.

Unless stated otherwise, percentages indicating the extent to which a particular ingredient is comprised in a mixture or composition refer to weight/weight (w/w) percentages. Similarly, unless stated otherwise, ratios specified herein are weight ratios.

Unless stated otherwise, the designation of a range in the present application using a hyphen (“-”) separating two bracketing values X and Y, or two bracketing ratios, is to be understood as meaning and disclosing the specified range in which both endpoint values X and Y are included. The same applies to a range expressed as “from X to Y”. Accordingly, the expressions of ranges as “X-Y”, “of X to Y”, “from X to Y”, “of X-Y” and “from X-Y” are to be understood equivalently as meaning and disclosing a range encompassing the end value X, all values between X and Y, as well as the end value Y.

As used herein the term “about” when referring to a particular value, e.g. an endpoint or endpoints of a range, encompasses and discloses, in addition to the specifically recited value itself, a certain variation around that specifically recited value. Such a variation may for example arise from normal measurement variability, e.g. in the weighing or apportioning of various substances by methods known to the skilled person. The term “about” shall be understood as encompassing and disclosing a range of variability above and below an indicated specific value, said percentage values being relative to the specific recited value itself, as follows: The term “about” may encompass and disclose variability of ±5.0%. The term “about” may encompass and disclose variability of ±4.5%. The term “about” may encompass and disclose variability of ±4.0%. The term “about” may encompass and disclose variability of ±3.5%. The term “about” may encompass and disclose variability of ±3.0%. The term “about” may encompass and disclose variability of ±2.5%. The term “about” may encompass and disclose variability of ±2.0%. The term “about” may encompass and disclose variability of ±1.5%. The term “about” may encompass and disclose variability of ±1.0%. The term “about” may encompass and disclose variability of +0.5%. The term “about”, in reference to the particular recited value, may encompass and disclose that exact particular value itself, irrespective of any explicit mention that this exact particular value is included; even in the absence of an explicit indication that the term “about” includes the particular exact recited value, this exact particular value is still included in the range of variation created by the term “about”, and is therefore disclosed in the present application. Unless stated otherwise, where the term “about” is recited before the first endpoint of a numerical range, but not before the second endpoint of that range, this term, and the variability it implies in scope and disclosure, refers to both the first endpoint of the range and the second endpoint of the range. For instance, a recited range of “about X to Y” should be read as “about X to about Y”. The same applies for a recited range of ratios. For instance, a recited range of weight ratios of “about X:Y-A:B” should be read as a weight ratio of “(about X):(about Y)-(about A):(about B)”.

As used herein, the term “a combination thereof” denotes any combination thereof, in any proportion. That is, the term encompasses any combination of the listed features (e.g. a combination of any two listed features, any three listed features, etc.) in any relative amounts. The skilled person can readily determine which amounts may be used in line with the intention of the invention.

As used herein, the term “for oral delivery” denotes that the product is adapted for delivery of the active ingredient in the oral cavity of a user.

As used herein, the term “orally dissolvable” denotes a compound or a composition which is fully dissolvable under the conditions existing in a user's mouth.

As used herein, the term “active ingredient” denotes a substance that has a modulating, e.g. a stimulating effect on the nervous system of a user. In the present invention, the active ingredient is selected from the group consisting of nicotine, caffeine, anatabine, cannabinoids and a combination thereof in any proportion.

As used herein, the term “free of active ingredient” denotes the detectable absence of an active ingredient.

As used herein, the term “nicotine” denotes a nicotine source. The nicotine source can be any nicotinic compound. Such nicotinic compound includes nicotine in free base form (i.e. nicotine in a non-protonated form), salt form, as a complex, or as a solvate. An example of a nicotine complex is a resin complex of nicotine, where nicotine is bound in an ion exchange resin, such as nicotine polacrilex. Examples of nicotine salts are hydrochloride, dihydrochloride, monotartrate, bitartrate, sulfate, salicylate, citrate, stearate, glutarate, aspartate, palmitate, and lactate. A combination thereof in any proportion is also possible and disclosed. Further possible forms of nicotine are discussed below.

As used herein “nicotine content” denotes the content of the nicotine source calculated as free base nicotine in weight/weight (w/w) percentage, also referred to herein as “wt %”.

As used herein, the term “cannabinoid” refers to a class of diverse chemical compounds that acts on cannabinoid receptors, also known as the endocannabinoid system, in cells that alter neurotransmitter release in the brain. Ligands for these receptor proteins include the endocannabinoids produced naturally in the body by animals; phytocannabinoids, found in cannabis; and synthetic cannabinoids, manufactured artificially. Cannabinoids found in cannabis include, without limitation: cannabigerol (CBG), cannabichromene (CBC), cannabidiol (CBD), tetrahydrocannabinol (THC), cannabinol (CBN), cannabinodiol (CBDL), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM), cannabinerolic acid, cannabidiolic acid (CBDA), cannabinol propyl variant (CBNV), cannabitriol (CBO), tetrahydrocannabinolic acid (THCA), and tetrahydrocannabivarinic acid (THCV A). A combination thereof in any proportion is also possible and disclosed in the context of the present invention. As used herein, the term “mucoadhesive” denotes a natural or synthetic substance that can adhere to a subject's mucous membrane. Non-limiting examples of mucoadhesives include gelatin; starch; cellulose derivatives such as for example carboxymethyl cellulose (CMC), hydroxyethyl cellulose (HEC), hydroxypropyl cellulose (HPC), hydroxypropyl methylcellulose (HPMC), methylcellulose, sodium carboxymethyl cellulose (Na CMC); agarose; chitosan, including chitosan derivatives such as for example trimethyl chitosan (TMC) and chitosan glutamate; hyaluronic acid; polyvinylpyrrolidone (PVP); polyacrylates such as for example polyacrylic acid (PAA, e.g. Carbopol®), polyhydroxyethylmetacrlylate (PHEMA); polyvinylalcohol (PVA); one or more lectins such as for example proteins or glycoproteins, possibly derivatized by e.g. polyethylene glycol (PEG), PVA, PAA, e.g. Carbopol®, PHEMA, HEC, HPC, HPMC, methylcellulose, Na CMC; one or more thiolated polymers (“thiomers”); polyethylene glycol (PEG) (e.g. PEG-6 stearate and PEG-4000); sodium alginate; guar gum; karya gum; gellan gum; agar; retene; and tragacanth. A combination thereof in any proportion is also possible and disclosed.

As used herein, the term “bulk agent” denotes a substance whose content is 50 wt % or more of the backing layer. In the present invention, a bulk agent may be selected from the group consisting of mono-, di-, and polysaccharides and sugar alcohols. Non-limiting examples of bulk agents include glucose, fructose and galactose as possible monosaccharides; sucrose, lactose, maltose and trehalose as possible disaccharides; starch (e.g. cassava starch) as a possible polysaccharide; and xylitol, sorbitol, erythritol, maltitol, isomalt, lactitol and mannitol as possible sugar alcohols. A combination thereof in any proportion is also possible and disclosed.

As used herein, the term “flavoring agent” is any flavorful or aromatic substance capable of altering the sensory characteristics of a compound or a composition (e.g. the sensory characteristics associated with the backing layer). Examples of sensory characteristics that can be modified by the flavoring agent include taste (e.g. sweet, salty, sour, bitter, etc.), mouthfeel, moistness, coolness/heat, and/or fragrance/aroma. Flavoring agents may be natural or synthetic, and the character of the flavors imparted thereby may be described, without limitation, as fresh, sweet, salty, umami, herbal, confectionary, floral, fruity, or spicy. Specific types of flavors include, but are not limited to, vanilla, coffee, chocolate/cocoa, cream, mint, spearmint, menthol, peppermint, wasabi, azuki, ginseng, kelp, wintergreen, eucalyptus, lavender, cardamom, nutmeg, cinnamon, clove, cascarilla, sandalwood, honey, jasmine, ginger, anise, sage, licorice, lemon, orange, apple, peach, lime, cherry, strawberry, and any combination thereof in any proportion. Flavoring agents also include components that are considered moistening, cooling or smoothening agents, such as eucalyptus. The flavoring agent can mask the flavor of other components in a composition (e.g. bitterness). Non-limiting examples of masking agents include sweeteners and compounds which can encapsulate flavors (e.g. maltodextrin). The flavoring agent may comprise at least one volatile flavor component. As used herein, “volatile” refers to a chemical substance that forms a vapor readily at ambient temperatures (i.e., a chemical substance that has a high vapor pressure at a given temperature relative to a nonvolatile substance). Typically, a volatile flavor component has a molecular weight below about 400 Da, and often include at least one carbon-carbon double bond, carbon-oxygen double bond, or both. Examples of volatile flavor components include alcohols, aldehydes, aromatic hydrocarbons, ketones, esters, terpenes, terpenoids, trigeminal sensates, or a combination thereof. Non-limiting examples of aldehydes include vanillin, ethyl vanillin, p-anisaldehyde, hexanal, furfural, isovaleraldehyde, cuminaldehyde, benzaldehyde, and citronellal. Non-limiting examples of ketones include 1-hydroxy-2-propanone and 2-hydroxy-3methyl-2-cyclopentenone-1-one. Non-limiting examples of esters include allyl hexanoate, ethyl heptanoate, ethyl hexanoate, isoamyl acetate, and 3-methylbutyl acetate. Non-limiting examples of terpenes include sabinene, limonene, gamma-terpinene, beta-famesene, nerolidol, thujone, myrcene, geraniol, nerol, citronellol, linalool, and eucalyptol. Preferred flavoring agents include mint and fruit taste flavors. A combination thereof in any proportion is also possible and disclosed.

In the present invention, the system for oral delivery comprises a backing layer extending over the mucoadhesive layer. As used herein, the term “extending over” (or grammatically related variants thereof) means that the backing layer completely covers one of the surfaces of the mucoadhesive layer. The backing layer may also completely cover the sides of the mucoadhesive layer so that only the bottom surface (the surface which is in contact with the oral mucosa) of the mucoadhesive layer is exposed.

As used herein, the term “binder” denotes a substance which is different from the bulk agent and which gives structural integrity to the bulk agent (e.g. sucrose). Non-limiting examples of binders are (sodium) carboxymethyl cellulose ((Na) CMC), (sodium) alginate, polyvinyl alcohol, polyethylene glycol (PEG), for example PEG 6-stearate or PEG-4000, microcrystaline cellulose, hydroxyethyl cellulose, hydroxypropyl cellulose, methylcellulose, hydroxypropylmethyl cellulose, Carbopol®, guar gum, gelatin, karya gum, polyacrylic acid, acacia gum, and polyvinyl pyrrolidone. A combination thereof in any proportion is also possible and disclosed.

As used herein, the term “dissolution enhancer” denotes a substance that accelerates the dissolution (reduces the dissolution time) of the backing layer. Non-limiting examples of a dissolution enhancer include citric acid, sodium bicarbonate, agar, amorphous (non-crystalline) carbohydrates, e.g. amorphous (non-crystalline) sugar, sodium starch glycolate, crosscaramellose sodium, alginic acid, crospovidone, and calcium silicate. A combination thereof in any proportion is also possible and disclosed.

As used herein, the term “gelling agent” denotes a substance which, when mixed with water, can form a gel. Non-limiting examples of gelling agents include sorbitol, glycerol, microcrystalline cellulose, and acacia gum. A combination thereof in any proportion is also possible and disclosed.

As used herein, the term “penetration enhancer” denotes a substance that accelerates the absorption (reduces the absorption time) of the active ingredient through the oral mucosa membranes into the blood stream. Non-limiting examples of penetration enhancers include bile salts such as for example sodium glycodeoxycholate, one or more trihydroxy salt of sodium cholate, sodium glycocholate, sodium taurocholate, dihydroxy salt of sodium deoxycholate, sodium glycodeoxycholate and sodium taurodeoxycholate; one or more fatty acid (e.g., oleic acid, lauric acid, cod liver oil, and one or more of their salts and esters (e.g., sodium laurate, sodium caprate, glyceryl monostearate, one or more sucrose fatty acid ester, and diethylene glycol monoethyl ether); one or more azone such as for example laurocapram; one or more surfactant such as sodium dodecyl (lauryl) sulfate, one or more polysorbate, one or more laureth, one or more Brij and benzalkonium chloride; one or more complexing agent such as one or more cyclodextrin (e.g. 2-hydroxypropyl beta-cyclodextrin or poly-beta-cyclodextrin), sodium edetate, ethylenediaminetetraacetic acid (EDTA), and sodium citrateone or more co-solvent such as ethanol and propylene glycol; lecithin; and sodium fusidate. A combination thereof in any proportion is also possible and disclosed in the context of the present invention.

As used herein, the term “demulcent” denotes a substance that relieves irritation of the mucous membranes in the mouth by forming a protective film.

### System for Oral Delivery of an Active Ingredient

The present invention provides a system for oral delivery of an active ingredient, the system comprising:
a) a mucoadhesive layer comprising a mucoadhesive and an active ingredient and
b) a backing layer extending over the mucoadhesive layer, said backing layer comprising a bulk agent and optionally a flavoring agent,
wherein the active ingredient is selected from the group consisting of nicotine, caffeine, anatabine, at least one cannabinoid such as tetrahydrocannabinol, and a combination thereof,
wherein the delivery system is orally dissolvable, and
wherein the bulk agent is selected from the group consisting of at least one mono-, di-, and/or polysaccharide, at least one sugar alcohol, and a combination thereof.

The system for oral delivery of an active ingredient of the present invention is configured to allow efficient transmucosal delivery of the active ingredient. Furthermore, the delivery system is orally dissolvable.

The system comprises a mucoadhesive layer and a backing layer. The active ingredient is contained in the mucoadhesive layer. The backing layer is typically free of active ingredient and extends over the mucoadhesive layer. The backing layer is water permeable. During use, the system is placed in the user's mouth such that the mucoadhesive layer is in contact with the oral mucosa. The mucoadhesive layer can securely adhere to the oral mucosa, thereby avoiding swallowing of the active ingredient. The backing layer covers the surface of the mucoadhesive layer opposite the surface which is in contact with the oral mucosa. The backing layer thus prevents contact of the active ingredient in the mucoadhesive layer with saliva and ensures that the active ingredient is not swallowed. Rather, the active ingredient is advantageously absorbed directly into the bloodstream directly through the oral mucosa.

The bulk agent provides taste (e.g. sweetness) and gradually dissolves over time. The thickness of the backing layer and its composition can be chosen to match the dissolution time of the mucoadhesive layer. In order to avoid contact of the active ingredient with the saliva, the dissolution time of the backing layer should be the same or slightly longer (e.g. 10% longer) than the dissolution time of the mucoadhesive layer. The skilled person is familiar with the dissolution profiles of the compounds making up the backing layer and the mucoadhesive layer of the present invention and can easily adjust the thicknesses and compositions of these layers to ensure a suitable match of dissolution times.

The system for oral delivery of the present invention can comprise additional layers, provided that such layers do not impair the function of the mucoadhesive layer and the backing layer. For example, a layer formed of a gelling agent can be present between the mucoadhesive layer and the backing layer. Alternatively or in addition, one or more coating layers can be formed on the surface of the backing layer opposite the mucoadhesive layer.

### a) Mucoadhesive Layer

The mucoadhesive layer adheres to the oral mucosa of the user. This ensures a prolonged contact between the oral mucosa and the active ingredient and avoids contact of the active ingredient with saliva. As a result, the active ingredient is efficiently delivered through the mucosal membranes in the user's mouth. Delivery proceeds efficiently into the bloodstream rather than inefficiently through the gastrointestinal tract, as is the case when the active ingredient is swallowed.

The mucoadhesive layer comprises a mucoadhesive. Preferably, the mucoadhesive is selected from the group consisting of gelatin; pectin; starch; cellulose derivatives such as for example carboxymethyl cellulose (CMC), hydroxyethyl cellulose (HEC), hydroxypropyl cellulose (HPC), hydroxypropyl methylcellulose (HPMC), methylcellulose, sodium carboxymethyl cellulose (Na CMC); agarose; chitosan including chitosan derivatives such as for example trimethyl chitosan (TMC) and chitosan glutamate; hyaluronic acid; polyvinylpyrrolidone (PVP); polyacrylates such as for example polyacrylic acid (PAA, e.g. Carbopol®); polyhydroxyethylmetacrlylate (PHEMA); polyvinylalcohol (PVA); one or more lectins such as for example proteins or glycoproteins; one or more thiolated polymers (“thiomers”); polyethylene glycol (PEG); sodium alginate; guar gum; karya gum; gellan gum; agar; retene; tragacanth; and a combination thereof.

Combinations of mucoadhesives can be used to obtain a mucoadhesive layer with good properties (adhesive strength, gel strength and oral solubility).

In a preferred embodiment, the mucoadhesive is selected from the group consisting of gelatin, chitosan, (sodium) carboxymethyl cellulose, hydroxypropylmethyl cellulose, polyvinyl alcohol, gellan gum, agar, agarose, and a combination thereof. These mucoadhesives are advantageous in terms of adhesive strength and disintegration time. That is, these mucoadhesives have excellent adhesive strength and retain the active ingredient long enough for it to be transmucosally delivered. At the same time, these mucoadhesives have excellent oral solubility. More preferably, the mucoadhesive is selected from the group consisting of gelatin, (sodium) carboxymethyl cellulose, hydroxypropylmethyl cellulose, and a combination thereof. In a preferred embodiment, the mucoadhesive is a combination of CMC and gelatin. In another preferred embodiment, the mucoadhesive is HPMC, Na CMC, gellan gum, chitosan, agarose, or a combination thereof.

In order to further improve the properties of the mucoadhesive layer (adhesive strength, gel strength, oral solubility, elastic properties, density, color, etc.), the mucoadhesive layer may also contain a gelling agent. In a preferred embodiment, the gelling agent is selected from the group consisting of sorbitol, glycerol, microcrystalline cellulose, and acacia gum.

In a further preferred embodiment, the mucoadhesive is HPMC and the gelling agent is glycerol. In the above embodiment, the weight ratio of HPMC to glycerol is preferably from about 1:5 to about 5:1, more preferably from about 1:2 to about 2:1, and the weight ratio of water to HPMC is preferably from about 50:1 to about 10:1, more preferably from about 30:1 to about 15:1.

The mucoadhesive layer is prepared by mixing the mucoadhesive, active ingredient and optional ingredients (e. g, gelling agent, penetration enhancer, etc.) with water. The components of the mucoadhesive layer are dissolved in water. The dissolution can be achieved or accelerated by heating the mixture. That is, the mixture can be heated to dissolve its components completely. Heating can also be used to increase the viscosity of the mixture. Once the ingredients have dissolved in water, the obtained paste/gel is cast on a plate (e.g. a metal plate) and allowed to dry to obtain a mucoadhesive layer.

The mucoadhesive layer has a total content of water, mucoadhesive and gelling agent of at least about 50 wt % based on the total weight of the mucoadhesive layer, preferably at least about 60 wt %, more preferably at least about 70 wt %, most preferably at least about 80 wt %, based on the total weight of the mucoadhesive layer.

The mucoadhesive layer can be in the form of a film having an area of about 0.1 cm²-5 cm², preferably about 0.5 cm²-3 cm², more preferably about 0.8 cm²-2 cm², and most preferably about 0.9 cm²-1.5 cm². In a preferred embodiment, in combination with the above or below embodiments, the film has a thickness of about 0.1 mm-10 mm, preferably about 0.5 mm-5 mm, more preferably about 0.7 mm-3 mm, and most preferably about 1 mm-2 mm. The shape of the mucoadhesive layer is not particularly limited. In a preferred embodiment, the mucoadhesive layer has a generally oval shape (e.g. round shape) or a generally rectangular shape (e.g. square shape).

The mucoadhesive layer comprises an active ingredient selected from the group consisting of nicotine, caffeine, anatabine, at least one cannabinoid such as tetrahydrocannabinol, and/or a combination thereof in any proportion. The weight ratio of active ingredient to mucoadhesive in the mucoadhesive layer is preferably about 1:1 to 1:1000, more preferably about 1:20 to 1:500, even more preferably about 1:50 to 1:200, and most preferably about 1:80 to 1:150.

In a preferred embodiment, the active ingredient is nicotine and the content thereof in the mucoadhesive layer is about 1 wt %-20 wt %, preferably about 2 wt %-15 wt %, more preferably about 5 wt %-12 wt %, and most preferably about 6 wt %-10 wt % based on the total weight of the backing layer.

In a preferred embodiment, the nicotine is a nicotine salt. The nicotine salt may be generated in situ during the preparation of the mucoadhesive layer by mixing nicotine free base with an acidic counterpart (e.g. citric acid).

In a further preferred embodiment, the nicotine can be a liquid form of nicotine (e.g. nicotine free base) which is encapsulated (e. g. by means of cyclo-dextrins).

In a preferred embodiment, the nicotine is selected from the group consisting of nicotine bitartrate, nicotine polacrilex, nicotine betadex (beta-cyclodextrin), nicotine o-salicylate, nicotine gentisate, nicotine gallate, nicotine phthalate, nicotine isophthalate, nicotine-acesulfame H, nicotine benzoate, nicotine fumarate, nicotine R-mandelate, nicotine hydrochlorate, nicotine L-lactate, nicotine levulinate, nicotine phosphorate, nicotine ascorbate, nicotine ferulate, nicotine vanilate, nicotine picolinate, nicotine cumate, nicotine nicotinate, nicotine pyruvate, nicotine gamma-resorcylate, and a combination thereof.

In a further preferred embodiment, the active ingredient is caffeine.

In a further preferred embodiment, the active ingredient is anatabine.

The mucoadhesive layer optionally comprises a penetration enhancer. The penetration enhancer improves the transfer of the active ingredient through the mucosa and thus reduces the absorption time of the active ingredient. Thus, if a faster absorption of the active ingredient is desired, a penetration enhancer can be added to the mucoadhesive layer. When a penetration enhancer is present, its content is typically about 1 wt %-5 wt % (preferably about 1 wt %-3 wt %, more preferably about 1.5 wt %-2 wt %) based on the total weight of the mucoadhesive layer.

In a preferred embodiment, the penetration enhancer is selected from the group consisting of bile salts such as sodium glycodeoxycholate, trihydroxy salts sodium cholate, sodium glycocholate, sodium taurocholate, dihydroxy salt sodium deoxycholate, sodium glycodeoxycholate and sodium taurodeoxycholate; fatty acids (e.g., oleic acid, lauric acid, cod liver oil) and their salts and esters (e.g., sodium laurate, sodium caprate, glyceryl monostearate, sucrose fatty acid esters, and diethylene glycol monoethyl ether); azones such as laurocapram; surfactants such as sodium dodecyl (lauryl) sulfate, polysorbates, Brijs and benzalkonium chloride; complexing agents such as cyclodextrin, sodium edetate, ethylenediaminetetraacetic acid (EDTA), and sodium citrate; co-solvents such as ethanol and propylene glycol; lecithin; and sodium fusidate. In a more preferred embodiment, the penetration enhancer is selected from the group consisting of bile salts such as sodium glycodeoxycholate, trihydroxy salts sodium cholate, sodium glycocholate, sodium taurocholate, dihydroxy salt sodium deoxycholate, sodium glycodeoxycholate and sodium taurodeoxycholate, fatty acids (e.g., oleic acid, lauric acid, cod liver oil) and their salts and esters (e.g., sodium laurate, sodium caprate, glyceryl monostearate, sucrose fatty acid esters, and diethylene glycol monoethyl ether); and azones such as laurocapram. A combination of the above penetration enhancers can also be used.

The mucoadhesive layer may also comprise a demulcent. In a preferred embodiment, the demulcent is selected from the group consisting of at least one polysaccharide, acid, gum Arabic, phenol, honey, marshmallow (root), ghee, licorice, slippery elm, and at least one essential oil such as essential oil of sage, thyme, tee-tree, eucalyptus, oregano, chamomile, peppermint, ginger, Echinacea and/or silk-cotton seed oil, and a combination thereof. In another preferred embodiment, in optional combination with the above or below embodiments, the demulcent content is about 1 wt %-10 wt %, preferably about 1 wt %-7 wt %, more preferably about 2 wt %-5 wt %, and most preferably about 2 wt %-4 wt % based on the total weight of the mucoadhesive layer.

The mucoadhesive layer may further comprise one or more pain relieving agent such as menthol, capsaicin (e.g. from Cayenne pepper), oleocanthal, sodium bicarbonate, butter, phenol, Arkovox® (Arkopharma), amylmetacresol, dichlorobenzyl alcohol, hexylresorcinol, benzocaine, dextromethorphan, lidocaine, paracetamol, and ibuprofen, or any combination thereof.

In a preferred embodiment, the total amount of demulcent and a pain relieving agent in the mucoadhesive layer is about 1-10 wt %, preferably about 1-7 wt %, more preferably about 2-5 wt %, and most preferably about 2-4 wt % based on the total weight of the mucoadhesive layer.

The mucoadhesive layer may also comprise a dissolution enhancer. The content of the dissolution enhancer in the mucoadhesive layer is about 0 wt %-20 wt % of the total weight of the mucoadhesive layer, preferably about 1 wt %-15 wt % of the total weight of the mucoadhesive layer, more preferably about 2 wt %-10 wt % of the total weight of the mucoadhesive layer, and most preferably about 3 wt %-5 wt % based on the total weight of the mucoadhesive layer. The dissolution enhancer reduces the dissolution time of the mucoadhesive layer.

### b) Backing Layer

The system for oral delivery of the present invention comprises a backing layer extending over the mucoadhesive layer. The backing layer comprises a bulk agent and optionally a flavoring agent. The bulk agent is selected from the group consisting of mono-, di-, and polysaccharides, sugar alcohols, and combinations thereof in any proportion. In a preferred embodiment, the bulk agent is selected the group consisting of glucose, fructose, galactose, sucrose, lactose, maltose, trehalose, starch, xylitol, sorbitol, erythritol, mannitol, and a combination thereof. In another preferred embodiment, the bulk agent is xylitol, erythritol, or a combination thereof. In a further preferred embodiment, the bulk agent is a combination of xylitol and erythritol in a weight ratio of about 1:5 to 5:1, preferably about 1:2 to 2:1, more preferably about 1:1.5 to 1.5:1, most preferably about 1:1.

Since the backing layer extends over the mucoadhesive layer, it provides a physical barrier between the active ingredient in the mucoadhesive layer and saliva and thus the contact of active ingredient present in the mucoadhesive layer with saliva can be avoided. Thus, swallowing of the active ingredient is avoided and the active ingredient migration through the transmucosal route is increased. The transmucosal route allows more efficient delivery of the active ingredient than does the gastrointestinal route.

The backing layer may consist of the bulk agent. The bulk agent may be in a compressed form (e.g. in tablet form). Since the bulk agent generally has a sweet flavor, the backing layer also improves the user experience of the system by providing a pleasant sweet flavor while the inventive system for oral delivery is in the oral cavity. The bulk agent is orally dissolvable and disintegrates in the mouth of the user.

The backing layer optionally comprises a flavoring agent. The flavoring agent can be vanilla, coffee, chocolate/cocoa, cream, mint, spearmint, menthol, peppermint, wintergreen, eucalyptus, lavender, cardamom, nutmeg, cinnamon, clove, cascarilla, sandalwood, honey, jasmine, ginger, anise, sage, licorice, lemon, orange, apple, peach, lime, cherry, strawberry, or a combination thereof. The content of flavoring agent in the backing layer is typically 0 wt %-30 wt %, preferably about 1 wt %-20 wt %, more preferably about 5 wt %-15 wt %, and most preferably about 7 wt %-12 wt % based on the total weight of the backing layer. Also, small amounts (about 0 wt %-2 wt % of the total weight of the backing layer) of nicotine (e.g. a nicotine salt such as nicotine stearate or palmitate) can be added to the backing layer to impart a nicotine taste to the backing layer.

In a preferred embodiment, the flavoring agent is in a powder (solid) form. In another preferred embodiment, the flavoring agent is an encapsulated liquid aroma.

The backing layer may comprise a binder. Depending on the bulk agent used, the addition of a binder can improve the mechanical strength of the backing layer. Furthermore, the addition of a binder allows the backing layer to be prepared without applying excessive mechanical pressure. As a result, a less dense backing layer can be obtained which can disintegrate faster in the user's mouth. The binder can also improve the bonding between the backing layer and the mucoadhesive layer. The content of the binder in the backing layer is about 0 wt %-20 wt % based on the total weight of the backing layer, preferably about 1 wt %-15 wt % based on the total weight of the backing layer, more preferably about 2 wt %-10 wt % based on the total weight of the backing layer, and most preferably about 3 wt %-5 wt % based on the total weight of the backing layer.

In a preferred embodiment, the binder may be selected from the group consisting of (sodium) carboxymethyl cellulose, guar gum, gelatin, (sodium) alginate, polyvinyl alcohol, polyethylene glycol, microcrystaline cellulose, hydroxyethyl cellulose, hydroxypropyl cellulose, methylcellulose, hydroxypropylmethyl cellulose, hydroxypropyl methyl cellulose, Carbopol®, karya gum, polyacrylic acid, acacia gum, polyvinyl pyrrolidone, and any combination thereof. These binders can reduce the dissolution time of the backing layer to about 15-45 min (e.g. about 20-30 min), which is highly desirable.

The backing layer may also comprise a dissolution enhancer. The content of the dissolution enhancer in the backing layer is about 0 wt %-20 wt % of the total weight of the backing layer, preferably about 1 wt %-15 wt % of the total weight of the backing layer, more preferably about 2 wt %-10 wt % of the total weight of the backing layer, and most preferably about 3 wt %-5 wt % based on the total weight of the backing layer. The dissolution enhancer reduces the dissolution time of the backing layer. A dissolution enhancer can be present in the backing layer instead of, or in addition to, the binder. In a preferred embodiment, the backing layer comprises a binder and a dissolution enhancer, wherein the total weight of the binder and the dissolution enhancer in the backing layer is about 2 wt %-20 wt %, preferably about 5 wt %-15 wt %, more preferably about 7 wt %-10 wt % based on the total weight of the backing layer.

In a preferred embodiment, in optional combination with any of the above or below embodiments, the dissolution enhancer is selected from citric acid, sodium bicarbonate, sodium starch glycolate, crosscaramellose sodium, alginic acid, crospovidone, calcium silicate, or any combination thereof. The backing layer has an area which is at least equal to the area of the mucoadhesive layer. Preferably, the area of the backing layer is about 0-20% larger than the area of the mucoadhesive layer. More preferably, the area of the backing layer is about equal to the area of the mucoadhesive layer.

The thickness of the backing layer can be suitably selected based on the thickness of the mucoadhesive layer so as to ensure that the active ingredient is fully absorbed before the backing layer is dissolved. In one embodiment, the thickness of the backing layer is about 10% to about 500% larger than the thickness of the mucoadhesive layer, e.g., about 20% to about 200% larger.

### Preparation of the System for Oral Delivery of the Present Invention

The system for oral delivery of the present invention can be prepared by the following method:

A backing layer is prepared by mixing all components of the backing layer and pressing the mixture into a mould to obtain a backing layer with a desired shape.

A mucoadhesive layer is prepared as described herein. The surface of the mucoadhesive layer is wetted to create a gluing effect such that the backing layer can be disposed on the mucoadhesive layer and fixed thereon.

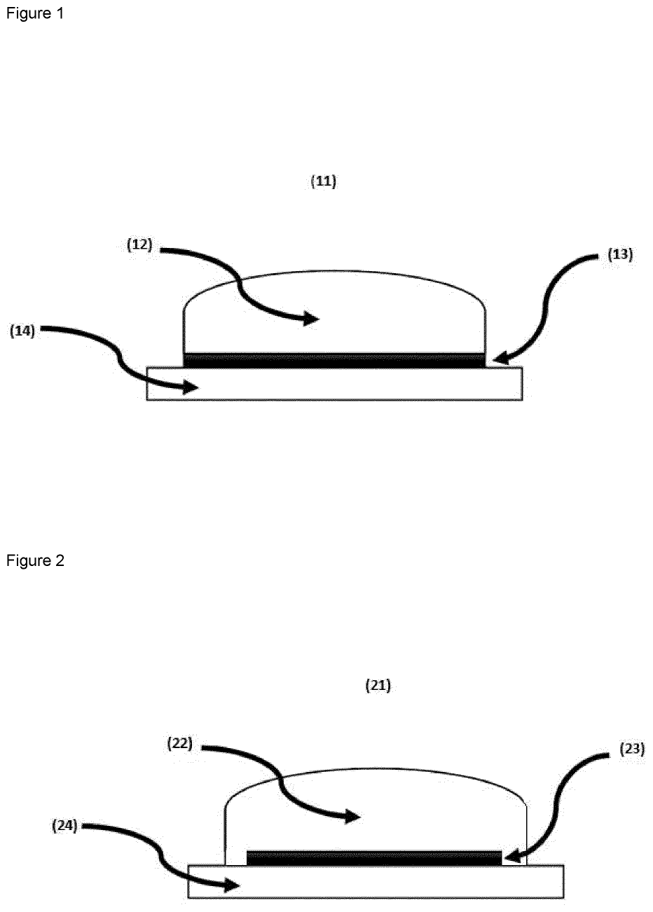

FIG. 1 shows one embodiment of the system of the present invention. The system (11) comprises a backing layer (12) made of pressed powder mix (a tablet) and a mucoadhesive film (3). The mucoadhesive film (13) contains active ingredient. The backing layer (12) extends over the top surface of the mucoadhesive layer (13). The system (11) is placed in the user's mouth so that the exposed surface of the mucoadhesive film (13) is in contact with the gingival mucosa (14).

During use, the system (11) is placed in the user's mouth such that the mucoadhesive film is in contact with the gingival mucosa (14). The mucoadhesive film (13) securely adheres to the gingival mucosa, thereby avoiding swallowing of the active ingredient. The backing layer (12) covers the surface of the mucoadhesive film (13) opposite the surface which is in contact with the gingival mucosa. The backing layer (12) thus prevents contact of the active ingredient in the mucoadhesive layer (13) with saliva and ensures that the active ingredient is not swallowed. Rather, the active ingredient is advantageously absorbed directly into the bloodstream directly through the oral mucosa.

FIG. 2 shows another embodiment of the system of the present invention. The system (21) comprises a backing layer (22) made of pressed powder mix (a tablet) and a mucoadhesive film (23). The mucoadhesive film (23) contains active ingredient. The backing layer (22) completely covers all three sides of the mucoadhesive layer (23) (top, left and right side in FIG. 2) so that only the bottom side (side which is in contact with the gingival mucosa (24)) of the mucoadhesive layer (23) is exposed. The system (21) is placed in the user's mouth so that the mucoadhesive film (23) is in contact with the gingival mucosa (24).

In an embodiment, the mucoadhesive is selected from the group consisting of gelatin, (sodium) carboxymethyl cellulose, hydroxypropylmethyl cellulose, and a combination thereof and the active ingredieint is nicotine, wherein the content of the mucoadhesive is 60 wt %-80 wt % based on the total weight of the mucoadhesive layer and the nicotine content is 5 wt %-12 wt %, based on the total weight of the mucoadhesive layer, the bulk agent is xylitol, erythritol, or a combination thereof and the flavoring agent is vanilla, coffee, chocolate/cocoa, cream, mint, spearmint, menthol, peppermint, or a combination thereof.

In a further embodiment, the mucoadhesive is selected from the group consisting of gelatin, (sodium) carboxymethyl cellulose, hydroxypropylmethyl cellulose, and a combination thereof and the active ingredient is a nicotine salt selected from the group consisting of nicotine lactate, nicotine aspartate, nicotine glutarate, nicotine citrate and a combination thereof, wherein the content of the mucoadhesive is about 60 wt %-80 wt % based on the total weight of the mucoadhesive layer and the nicotine content is about 5 wt %-10 wt %, based on the total weight of the mucoadhesive layer, the bulk agent is a combination of xylitol and erythritol and the flavoring agent is coffee, chocolate/cocoa, peppermint, or a combination thereof.

In a further embodiment, in combination with any of the above embodiments, the mucoadhesive layer comprises a pain relieving agent selected from menthol, capsaicin, oleocanthal, sodium bicarbonate, phenol, Arkovox®, amylmetacresol, dichlorobenzyl alcohol, hexylresorcinol, benzocaine, dextromethorphan, lidocaine, paracetamol, and ibuprofen, or any combination thereof and a demulcent selected from sage oil, thyme, tee-tree oil, eucalyptus oil, oregano oil, chamomile oil, peppermint oil, or a combination thereof, wherein the total amount of demulcent and a pain relieving agent in the mucoadhesive layer is about 2 wt %-5 wt %, based on the total weight of the mucoadhesive layer.

### Dosage Forms

In another aspect, the present invention relates to dosage forms comprising the system for oral delivery of the present invention. In a preferred embodiment, the dosage form is a tablet. The shape of the tablet is not particularly limited. In a preferred embodiment, the tablet has a generally round, e.g. oval or circular shape. In another preferred embodiment, in combination with any of the above or below embodiments, the dosage form is packaged in a blister pack. In another preferred embodiment, the dose of the active ingredient in the dosage form is about 0.1 g-5 g, preferably about 0.5 g-2 g, more preferably about 0.7 g-1.2 g.

The present invention is also directed to the following embodiments.

Embodiment 1. A system for oral delivery of an active ingredient, the system comprising:
a) a mucoadhesive layer comprising a mucoadhesive and an active ingredient and
b) a backing layer extending over the mucoadhesive layer, said backing layer comprising a bulk agent and optionally a flavoring agent,
wherein the active ingredient is selected from the group consisting of nicotine, caffeine, anatabine, at least one cannabinoid such as tetrahydrocannabinol, and a combination thereof,
wherein the delivery system is orally dissolvable, and
wherein the bulk agent is selected from the group consisting of at least one mono-, di-, and/or polysaccharide, at least one sugar alcohol, and a combination thereof.

Embodiment 2. The system according to embodiment 1, wherein the backing layer further comprises a binder.

Embodiment 3. The system according to Embodiment 2, wherein the binder is selected from the group consisting of (sodium) carboxymethyl cellulose, guar gum, gelatin, (sodium) alginate, polyvinyl alcohol, polyethylene glycol, microcrystaline cellulose, hydroxyethyl cellulose, hydroxypropyl cellulose, methylcellulose, hydroxypropylmethyl cellulose, hydroxypropyl methyl cellulose, Carbopol®, karya gum, polyacrylic acid, acacia gum, polyvinyl pyrrolidone, and any combination thereof.

Embodiment 4. The system according to Embodiment 2 or 3, wherein the content of the binder in the backing layer is about 1 wt %-15 wt % based on the total weight of the backing layer, preferably about 2 wt %-10 wt % based on the total weight of the backing layer, and more preferably about 3 wt %-5 wt % based on the total weight of the backing layer.

Embodiment 5. The system according to any one of Embodiments 1-4, wherein the backing layer further comprises a dissolution enhancer.

Embodiment 6. The system according to Embodiment 5, wherein the content of the dissolution enhancer in the backing layer is about 1 wt %-15 wt % of the total weight of the backing layer, preferably about 2 wt %-10 wt % of the total weight of the backing layer, and more preferably about 3 wt %-5 wt % based on the total weight of the backing layer.

Embodiment 7. The system according to Embodiment 1 or 3, wherein the backing layer comprises a binder and a dissolution enhancer, wherein the total weight of the binder and the dissolution enhancer in the backing layer is about 2 wt %-20 wt %, preferably about 5 wt %-15 wt %, more preferably about 7 wt %-10 wt % based on the total weight of the backing layer.

Embodiment 8. The system according to any one of Embodiments 5-7, wherein the dissolution enhancer is selected from the group consisting of citric acid, sodium bicarbonate, sodium starch glycolate, crosscaramellose sodium, alginic acid, crospovidone, calcium silicate, and a combination thereof.

Embodiment 9. The system according to any one of Embodiments 1-8, wherein the mucoadhesive is selected from the group consisting of gelatin; pectin; starch; cellulose derivatives such as for example carboxymethyl cellulose (CMC), hydroxyethyl cellulose (HEC), hydroxypropyl cellulose (HPC), hydroxypropyl methylcellulose (HPMC), methylcellulose, sodium carboxymethyl cellulose (Na CMC); agarose; chitosan including chitosan derivatives (e.g. trimethyl chitosan (TMC) or chitosan glutamate); hyaluronic acid; polyvinylpyrrolidone (PVP); polyacrylates (e.g. polyacrylic acid (PAA, e.g. Carbopol®)); polyhydroxyethylmetacrlylate (PHEMA); polyvinylalcohol (PVA); one or more lectins (e.g. proteins or glycoproteins); one or more thiolated polymers; polyethylene glycol (PEG); sodium alginate; guar gum; karya gum; gellan gum; agar; retene; tragacanth; and a combination thereof.

Embodiment 10. The system according to Embodiment 9, wherein the mucoadhesive is selected from the group consisting of gelatin, chitosan, (sodium) carboxymethyl cellulose, hydroxypropylmethyl cellulose, polyvinyl alcohol, gellan gum, agar, agarose, and a combination thereof, preferably, the mucoadhesive is selected from the group consisting of gelatin, (sodium) carboxymethyl cellulose, hydroxypropylmethyl cellulose, and a combination thereof.

Embodiment 11. The system according to Embodiment 10, wherein the mucoadhesive is a combination of CMC and gelatin.

Embodiment 12. The system according to Embodiment 10, wherein the mucoadhesive is HPMC, Na CMC, gellan gum, chitosan, agarose, or a combination thereof.

Embodiment 13. The system according to any one of Embodiments 1-12, wherein the mucoadhesive layer further comprises a gelling agent.

Embodiment 14. The system according to Embodiment 14, wherein the gelling agent is selected from the group consisting of sorbitol, glycerol, microcrystalline cellulose, and acacia gum. Embodiment 15. The system according to Embodiment 13 or 14, wherein the mucoadhesive is HPMC and the gelling agent is glycerol.

Embodiment 16. The system according to Embodiment 15, wherein the weight ratio of HPMC to glycerol is from about 1:5 to about 5:1, preferably from about 1:2 to about 2:1, and the weight ratio of water to HPMC is from about 50:1 to about 10:1, preferably from about 30:1 to about 15:1.

Embodiment 17. The system according to any one of Embodiments 1-16, wherein the mucoadhesive layer has a total content of water, mucoadhesive and gelling agent of at least about 50 wt % based on the total weight of the mucoadhesive layer, preferably at least about 60 wt %, more preferably at least about 70 wt %, most preferably at least about 80 wt %, based on the total weight of the mucoadhesive layer.

Embodiment 18. The system according to any one of Embodiments 1-17, wherein the mucoadhesive layer comprises an active ingredient selected from the group consisting of nicotine, caffeine, anatabine, at least one cannabinoid such as tetrahydrocannabinol, and a combination thereof.

Embodiment 19. The system according to Embodiment 18, wherein the weight ratio of active ingredient to mucoadhesive in the mucoadhesive layer is about 1:1 to 1:1000, preferably about 1:20 to 1:500, more preferably about 1:50 to 1:200, and most preferably about 1:80 to 1:150.

Embodiment 20. The system according to any one of embodiments 1-19, wherein the active ingredient is nicotine.

Embodiment 21. The system according to Embodiment 20, wherein the nicotine content of the mucoadhesive layer is about 1 wt %-20 wt %, preferably about 2 wt %- 15 wt %, more preferably about 5 wt %-12 wt %, and most preferably about 6 wt %-10 wt % based on the total weight of the backing layer.

Embodiment 22. The system according to Embodiment 20 or 21, wherein the nicotine is a nicotine salt.

Embodiment 23. The system according to Embodiment 20 or 21, wherein the nicotine is a liquid form of nicotine (e.g. nicotine free base) which is encapsulated (e. g. by means of cyclo-dextrins).

Embodiment 24. The system according to Embodiment 20 or 21, wherein the nicotine is selected from the group consisting of nicotine bitartrate, nicotine betadex (beta-cyclodextrin), nicotine o-salicylate, nicotine gentisate, nicotine gallate, nicotine phthalate, nicotine isophthalate, nicotine-acesulfame H, nicotine benzoate, nicotine fumarate, nicotine R-mandelate, nicotine hydrochlorate, nicotine L-lactate, nicotine levulinate, nicotine phosphorate, nicotine ascorbate, nicotine ferulate, nicotine vanilate, nicotine picolinate, nicotine cumate, nicotine nicotinate, nicotine pyruvate, nicotine gamma-resorcylate, and a combination thereof.

Embodiment 25. The system according to any one of Embodiments 1-19, wherein the active ingredient is caffeine.

Embodiment 26. The system according to any one of Embodiments 1-19, wherein the active ingredient is anatabine.

Embodiment 27. The system according to any one of Embodiments 1-26, wherein the mucoadhesive layer further comprises a penetration enhancer.

Embodiment 28. The system according to Embodiment 27, wherein the content of the penetration enhancer is about 1 wt %-5 wt %, preferably about 1 wt %-3 wt %, more preferably about 1.5 wt %-2 wt %, all ranges based on the total weight of the mucoadhesive layer.

Embodiment 29. The system according to Embodiment 27 or 28, wherein the penetration enhancer is selected from the group consisting of bile salts such as sodium glycodeoxycholate, trihydroxy salts sodium cholate, sodium glycocholate, sodium taurocholate, dihydroxy salt sodium deoxycholate, sodium glycodeoxycholate and sodium taurodeoxycholate; fatty acids (e.g., oleic acid, lauric acid, cod liver oil) and their salts and esters (e.g., sodium laurate, sodium caprate, glyceryl monostearate, sucrose fatty acid esters, and diethylene glycol monoethyl ether); azones such as laurocapram; surfactants such as sodium dodecyl (lauryl) sulfate, polysorbates, laureths, Brijs and benzalkonium chloride; complexing agents such as cyclodextrin, sodium edetate, ethylenediaminetetraacetic acid (EDTA), and sodium citrate; co-solvents such as ethanol and propylene glycol; lecithin; and sodium fusidate.

Embodiment 30. The system according to any one of Embodiments 1-29, wherein the mucoadhesive layer further comprises a demulcent.

Embodiment 31. The system according to Embodiment 30, wherein the demulcent is selected from the group consisting of at least one polysaccharide, galacturonic acid, gum Arabic, phenol, honey, marshmallow (root), ghee, licorice, slippery elm, and at least one essential oil such as essential oil of sage, thyme, tee-tree, eucalyptus, oregano, chamomile, peppermint, ginger, Echinacea and/or silk-cotton seed oil, and a combination thereof.

Embodiment 32. The system according to Embodiment 31 or 32, wherein the demulcent content is about 1 wt %-10 wt %, preferably about 1 wt %-7 wt %, more preferably about 2 wt %- 5 wt %, and most preferably about 2 wt %-4 wt %, all ranges based on the total weight of the mucoadhesive layer.

Embodiment 33. The system according to any one of Embodiments 1-32, wherein the mucoadhesive layer further comprises one or more pain relieving agent such as menthol, capsaicin (e.g. from Cayenne pepper), oleocanthal, sodium bicarbonate, butter, phenol, Arkovox® (Arkopharma), amylmetacresol, dichlorobenzyl alcohol, hexylresorcinol, benzocaine, dextromethorphan, lidocaine, paracetamol, and ibuprofen, or any combination thereof.

Embodiment 34. The system according to Embodiment 33, wherein the total amount of demulcent and a pain relieving agent in the mucoadhesive layer is about 1-10 wt %, preferably about 1-7 wt %, more preferably about 2-5 wt %, and most preferably about 2-4 wt %, all ranges based on the total weight of the mucoadhesive layer.

Embodiment 35. The system according to any one of Embodiments 1-34, wherein the mucoadhesive layer further comprises a dissolution enhancer.

Embodiment 36. The system according to Embodiment 35, wherein the content of the dissolution enhancer in the mucoadhesive layer is about 1 wt %- 20 wt % of the total weight of the mucoadhesive layer, preferably about 1 wt %-15 wt % of the total weight of the mucoadhesive layer, more preferably about 2 wt %-10 wt % of the total weight of the mucoadhesive layer, and most preferably about 3 wt %-5 wt % based on the total weight of the mucoadhesive layer.

Embodiment 37. The system according to any one of Embodiments 1-35, wherein the backing layer is free of active pharmaceutical ingredient.

Embodiment 38. The system according to any one of Embodiments 1-35, wherein the weight ratio of active ingredient in the mucoadhesive layer to the weight ratio of active ingredient in the backing layer is 1:1 to 1000:1.

Embodiment 39. The system according to any one of Embodiments 1-38, wherein the weight ratio of active ingredient to mucoadhesive in the mucoadhesive layer is about 1:1 to 1:1000. Embodiment 40. The system according to any one of Embodiments 1-39, wherein the backing layer further comprises a flavoring agent.

Embodiment 41. The system according to Embodiment 40, wherein the flavoring agent is vanilla, coffee, chocolate/cocoa, cream, mint, spearmint, menthol, peppermint, wintergreen, eucalyptus, lavender, cardamom, nutmeg, cinnamon, clove, cascarilla, sandalwood, honey, jasmine, ginger, anise, sage, licorice, lemon, orange, apple, peach, lime, cherry, strawberry, or a combination thereof.

Embodiment 42. The system according to Embodiment 40 or 41, wherein the content of flavoring agent in the backing layer is 0.5 wt %-30 wt %, preferably about 1 wt %-20 wt %, more preferably about 5 wt %-15 wt %, and most preferably about 7 wt %-12 wt %, all ranges based on the total weight of the backing layer.

Embodiment 43. the System According to Any One of Embodiments 40-42, Wherein the flavoring agent is in a powder (solid) form.

Embodiment 44. The system according to any one of Embodiments 40-42, wherein the flavoring agent is an encapsulated liquid aroma.

Embodiment 45. The system according to any one of Embodiments 1-44, wherein the backing layer has an area which is at least equal to the area of the mucoadhesive layer, preferably, the area of the backing layer is about 0-20% larger than the area of the mucoadhesive layer.

Embodiment 46. The system according to Embodiment 45, wherein the area of the backing layer is about equal to the area of the mucoadhesive layer.

Embodiment 47. The system according to any one of Embodiments 1-46, wherein the mucoadhesive layer has a single exposed surface and its remaining surfaces are covered by the backing layer.

Embodiment 48. A dosage form comprising a system according to any one of Embodiments 1-47.

Embodiment 49. The dosage form according to Embodiment 48, which is the form of a tablet. Embodiment 50. A blister pack comprising the dosage form according to Embodiment 48 or 49.

### EXAMPLES

### Example 1: Preparation of a Mucoadhesive Layer Nicotine/lactic acid solution was obtained by mixing 100 μl nicotine free base with 51 μl lactic acid (targeted molar ratio of 1:1).

Separately, 5 g of CMC was dissolved in 45 g water. Then, the nicotine/lactic acid solution was added to the CMC solution.

The obtained mixture was heated to dissolve all solids and obtain a gel. The obtained gel was cast on a plate and allowed to dry to obtain a mucoadhesive layer.

### Example 2: Preparation of a Mucoadhesive Layer

80 mg of aspartic acid was dissolved in 10 g of water by heating. 100 μl of nicotine was added to the aspartic acid solution.

Separately, 5 g of CMC was dissolved in 35 g water. Then, the nicotine/aspartic acid solution was added to the CMC solution and heated to obtain a gel. The obtained gel was cast on a plate and allowed to dry to obtain a mucoadhesive layer.

### Example 3: Preparation of a Backing Layer

A powder was prepared by mixing erythritol and xylitol in equal amounts and a flavor and an active ingredient (e.g. anatabine) were added. The mixture was pressed into a mould to obtain a desired shape (e.g. tablet) using a commercial apparatus (e.g. Table Press Flexitab S from Röltgen).

### REFERENCE SIGNS

11, 21 System for oral delivery
12, 22 Backing layer
13, 23 Mucoadhesive film
14, 24 Gingival mucosa

1.-12. (canceled)

13. A system for oral delivery of an active ingredient, the system comprising:
a) a mucoadhesive layer comprising a mucoadhesive and an active ingredient; and
b) a backing layer extending over the mucoadhesive layer, the backing layer comprising a bulk agent,
wherein the active ingredient is selected from the group consisting of nicotine, caffeine, anatabine, at least one cannabinoid such as tetrahydrocannabinol, and a combination thereof,
wherein the system is orally dissolvable, and
wherein the bulk agent is selected from the group consisting of at least one mono-, di-, and/or polysaccharide, at least one sugar alcohol, and a combination thereof.

14. The system according to claim 13, wherein the backing layer further comprises a flavoring agent.

15. The system according to claim 13, wherein the backing layer further comprises a binder.

16. The system according to claim 13, wherein the backing layer further comprises a dissolution enhancer.

17. The system according to claim 13, wherein the mucoadhesive layer further comprises a penetration enhancer.

18. The system according to claim 13, wherein the backing layer is free of active pharmaceutical ingredient.

19. The system according to claim 13, wherein the weight ratio of active ingredient in the mucoadhesive layer to a weight ratio of active ingredient in the backing layer is 1:1 to 1000:1.

20. The system according to claim 13, wherein a weight ratio of active ingredient to mucoadhesive in the mucoadhesive layer is about 1:1 to 1:1000.

21. The system according to claim 13, wherein the mucoadhesive is selected from the group consisting of gelatin, chitosan, (sodium) carboxymethyl cellulose, hydroxypropylmethyl cellulose, polyvinyl alcohol, gellan gum, agar, agarose, and a combination thereof.

22. The system according to claim 13, wherein the active ingredient is nicotine.

23. A dosage form comprising the system according to claim 13, which is the form of a tablet.

24. A blister pack comprising the dosage form according to claim 23.
